(19) United States
(12) Patent Application Publication
(10) Pub. No.: US 2007/0020333 A1
(43) Pub. Date: Jan. 25, 2007

(54) Controlled release of hypnotic agents

(76) Inventors: Chin-Chih Chiang, West Covina, CA (US); Ting-Wei Chang, Taipei (TW)

Correspondence Address: SHIHONG NICOLAOU, 4931 ARROYO LINDO AVE., SAN DIEGO, CA, 92117, US

(21) Appl. No.: 11/186,348

(22) Filed: Jul. 20, 2005

(51) Int. Cl. A61K 9/22; A61K 31/425

(52) U.S. Cl. 424/468; 514/373

(57) ABSTRACT

The application relates to the controlled release of a hypnotic agent with extended release profiles. The pharmaceutical composition and processes for manufacturing, and methods of using the controlled release formulation are provided.

### FIELD

The application relates to the controlled release of hypnotic drugs. The pharmaceutical compositions and methods of use for the oral administration of hypnotic drugs with extended release profiles are provided. The application further provides methods of preparing the formulations and the process for manufacturing.

### BACKGROUND

Various treatments for insomnia have been developed. Hypnotic drugs that have been approved by FDA include such as Ambien (zolpidem) which is based on the imidazopyridine backbone (see U.S. Pat. Nos. 4,382,938 and 4,460,592), Sonata (zaleplon) which is a pyrazolopyrimidine-based compound (see U.S. Pat. No. 4,626,538) and zopiclon (see U.S. Pat. No. 3,862,140).

Most of hypnotic drugs are marketed as immediate release oral dosage forms. In certain situations, the onset and duration of the hypnotic drugs are undesirable. For example, zolpidem is most effective when present in plasma within a certain concentration range. Above this range, there may be a danger that deleterious side effects may become manifest and even when there is not the danger, excess drug in the blood plasma may simply be wasted. As a result, the duration of action of the available zolpidem tablets is sometimes insufficiently short and thus does not accommodate a longer, uninterrupted and deep sleep. This disadvantage is also true for most other hypnotic agents that are used in immediate release oral dosage forms.

It is thus desirable to develop a pharmaceutical formulation for oral application of hypnotic agent that exhibits a fast and a prolonged action at the same time.

### SUMMARY

The application is directed to a pharmaceutical composition containing a hypnotic agent in a controlled-release dosage form that contains at least one immediate release element and at least one delayed release element. The hypnotic agents include zaleplon, zopiclon and zolpidem or a salt, solvate or hydrate thereof.

The controlled release dosage form can be a capsule, for example. The immediate release element contains hot-melt excipients that are dissolved in a media of pH<5. The hot-melt excipients are selected from Vitamin E TPGS, Gelucire, and Polyethylene glycols (PEGs), PEG-1500, Weobee and its derivatives, Witepsol and its derivatives, and Tweens, et al. The delayed release element contains ingredients of hot-melt excipients and pH sensitive materials that are dissolved in a media of pH≧5. These elements include such as sodium alginate, Eudragit, and Polyethylene glycols (PEGs), PEG 1500, and Gelucire, et al.

Another aspect of the invention is related to the method of treating sleeping disorders in a subject using the controlled release formulation of hypnotics.

Another aspect of the invention is also related to the method for the preparation of a controlled release capsule formulation of hypnotic agents. The method includes the steps of filling the capsule with at least one immediate release formulation element and at least one delayed release formulation element. The formulation element contains hot-melt excipients that are in liquid form at temperatures above 35° C.

### BRIEF DESCRIPTION OF THE DRAWINGS

FIG. 1. Dissolution Profiles of Zaleplon of Formulation 3

FIG. 2. Plasma Concentrations versus Time Profiles of Zaleplon for Sonata and Formulation 3

### DETAILED DESCRIPTIONS

The present disclosure relates to a controlled release of a hypnotic drug. Zaleplon is disclosed in U.S. Pat. No. 4,626,538; zopiclon is disclosed in U.S. Pat. No. 3,862,140; and zolpidem is disclosed in U.S. Pat. Nos. 4,382,938 and 4,460,592, and they are herein incorporated by reference in its entirety.

The controlled release formulation of the hypnotic agent is characterized by at least one immediate release formulation element and at least one delayed release formulation element. The formulation contains the hypnotic drug with a pharmaceutical acceptable excipient that melts at elevated temperature and become solid when stored at room temperatures. These pharmaceutical acceptable excipients not only are carriers for the hypnotic drug, but also may function as solubilizers or absorption enhancers for the hypnotic drug. The excipients are also capable of modifying the release profile of the hypnotic drug in the gastric intestinal tracts.

The immediate release formulation element comprises the hypnotic drug and a hot-melt excipient that can be dissolved in an acidic media (pH<5). Examples of hot melt excipients include such as Vitamin E TPGS, Gelucire, and Polyethylene glycols (PEGs), PEG-1500, Weobee and its derivatives, Witepsol and its derivatives, and Tweens. The percentage of hypnotic drug in an immediate release formulation can be varied from 1% to 40% (w/w). For example, an immediate release formulation of zaleplon may contain 4% (w/w) active and 96% (w/w) Vitamin E TPGS.

The delayed release formulation element comprises a hypnotic drug, hot-melt ingredients and excipients that can be dissolved in a media at pH≧5. Examples of such excipients include such as sodium alginate, Eudragit, and Polyethylene glycols (PEGs), PEG 1500, and Gelucire, et al. The percentage of hypnotic drug in the delayed release formulation can be varied from 1% to 40% (w/w). For example, a composition of the delayed release formulation of zaleplon contains 6% (w/w) active, 84% (w/w) PEG 1500, and 10% (w/w) Eudragit.

The controlled release formulations of a hypnotic drug can be prepared by using a hot-melt process. The process involves mixing the hypnotic drug with a pharmaceutical acceptable excipient that melts at elevated temperatures to form a liquid. The formulations become solid when stored at room temperatures. A two stage filling into capsules can be used, for example. The immediate release formulation element and the delayed release formulation element can be prepared separately. One of the formulation elements is first filled into the capsules in a liquid form. The liquid formulation is then cooled down to room temperatures into a solid form before the second formulation is filled in the same capsule. The formulation element having higher melting temperatures or higher viscosity is preferably to be filled first.

In general, the formulations contain ingredients that melt at elevated temperatures from about 35° C. to about 80° C. The hypnotic drug is then added into the melt and sufficiently mixed to form a homogeneous mixture during the preparation. Depending on the excipients, the temperatures, and the concentrations of the hypnotic drug, the mixture liquid can either be a solution, an emulsion or a suspension. This liquid can then be filled into capsules at elevated temperatures.

The performance of the controlled release formulation thus made can be evaluated by a standard dissolution method. The dissolution test of the formulation can be conducted by using the US Pharmacopoeia XXIII, Method I, in a basket apparatus at 50 rpm and temperatures at 37° C. The dissolution study is conducted in a dissolution medium of 900 mL of a simulated gastric fluid of 0.1N HCl (pH 1.0) for 2 hours initially. After 2 hours, the dissolution medium is decanted from the dissolution vessel and is replaced by a dissolution medium of 900 mL of simulated intestinal fluids (pH 7.2) for another 6 hours. The dissolved amounts of the hypnotic drug in the samples can be assayed by a HPLC method. The immediate release formulation element is dissolved in the more acidic environment, and the hypnotic drug is released rapidly in the simulated gastric fluids while the delayed-releasing formulation element remains intact. After the medium is changed into the simulated intestinal fluids, the delayed release formulation element is dissolved in the more basic medium and releases the hypnotic drug. Thus, an extended release profile of the controlled release formulation can be evaluated.

The controlled release formulations contain rapidly acting hypnotic drugs such as zaleplon, zopiclon and zolpidem, but not limited thereto. Other pharmaceutically active drugs can also be formulated into the controlled release formulations having the desired extended release profile as described in this disclosure. The controlled release formulations of the present application can be particularly useful in a method for inducing and maintaining sleep for the treatment of sleep disorders. The method is composed of an immediate release formulation element to induce the sleep and a delayed release formulation element to maintain the sleep. The controlled release formulations described in this disclosure have advantages by its modified release profiles and prolonged duration of the drug.

The disclosure is further illustrated by the following Examples.

### EXAMPLES

### Example 1

Compositions and Method of Preparation for Formulation 1

| | |
|---|---|
| Ingredient | Weight % |

Formulation 1a (delayed release element)

| | |
|---|---|
| Zaleplon | 6 |
| Gelucire | 54 |
| Sodium Alginate | 40 |
| TOTAL | 100 |

Formulation 1b (immediate release element)

| | |
|---|---|
| Zaleplon | 4 |
| Vitamin E TPGS | 96 |
| TOTAL | 100 |

Manufacturing Process of Formulation 1
Step A (Preparation of the Delayed Release Element):
1. Mixing 27 grams of Gelucire and 20 grams of sodium alginate in a suitable container. Maintaining the temperature at 70° C. during the mixing.
2. Sufficiently mixing the two excipients until homogeneous.
3. Adding 3 grams of zaleplon into the same container, and mixing until homogeneous.
4. Weighing 100 mg of the homogeneous suspension, and filling into a hard gelatin capsule.
5. Allowing the formulation to cool down to the room temperatures.
   Step B (Preparation of the Immediate Release Element):
6. Adding 48 grams of Vitamin E TPGS in a suitable container, and maintaining the temperature at 60° C.
7. Adding 2 grams of zaleplon into the same container. Maintaining the temperature at 60° C., and mixing the ingredients until homogeneous.
8. Weighing 100 mg of the homogeneous suspension, and filling into the same hard gelatin capsule.
9. Allowing the formulation to cool down to the room temperatures.

### Example 2

Compositions and Method of Preparation for Formulation 2

| | |
|---|---|
| Ingredient | Weight % |

Formulation 2a (delayed release element)

| | |
|---|---|
| Zaleplon | 6 |
| PEG 1500 | 84 |
| Eudragit | 10 |
| TOTAL | 100 |

Formulation 2b (immediate release element)

| | |
|---|---|
| Zaleplon | 4 |
| Vitamin E TPGS | 96 |
| TOTAL | 100 |

Manufacturing Process of Formulation 2
Step A (Preparation of the Delayed Release Element):
1. Mixing 42 grams of PEG 1500 and 5 grams of Eudragit in a suitable container. Maintaining the temperature at 70° C. during the mixing.
2. Sufficiently mixing the two excipients until homogeneous.
3. Adding 3 grams of zaleplon into the same container, and mixing until homogeneous.
4. Weighing 100 mg of the homogeneous liquid, and filling into a hard gelatin capsule.
5. Allowing the formulation to cool down to the room temperatures.
   Step B (Preparation of the Immediate Release Element):
6. Adding 48 grams of Vitamin E TPGS in a suitable container, and maintaining the temperature at 60° C.
7. Adding 2 grams of zaleplon into the same container. Maintaining the temperature at 60° C. Mixing the ingredients until homogeneous.
8. Weighing 100 mg of the homogeneous suspension, and filling into the same hard gelatin capsule.
9. Allowing the formulation to cool down to the room temperatures.

### Example 3

Compositions and Method of Preparation for Formulation 3

| | |
|---|---|
| Ingredient | Weight % |

Formulation 3a (delayed release element)

| | |
|---|---|
| Zaleplon | 3.3 |
| Gelucire | 44.7 |
| Sodium Alginate | 40.7 |
| PEG 1500 | 11.3 |
| TOTAL | 100 |

Formulation 3b (fast release element)

| | |
|---|---|
| Zaleplon | 3.3 |
| PEG 1500 | 96.7 |
| TOTAL | 100 |

Manufacturing Process of Formulation 3
Step A (Preparation of the Delayed Release Element):
1. Mixing 44.7 grams of Gelucire and 11.3 grams of PEG 1500 in a suitable container. Maintaining the temperature at 75° C. during the mixing.
2. Adding 40.7 grams of sodium alginate into the same container, sufficiently mixing all the excipients until homogeneous.
3. Adding 3.3 grams of zaleplon into the same container, and mixing with the other excipients until homogeneous.
4. Weighing 150 mg of the homogeneous suspension, and filling into a hard gelatin capsule.
5. Allowing the formulation to cool down to the room temperatures.
   Step B (Preparation of the Immediate Release Formulation):
6. Adding 96.7 grams of PEG 1500 in a suitable container, and maintaining the temperature at 45° C.
7. Adding 3.3 grams of zaleplon into the same container. Maintaining the temperature at 45° C. Mixing the ingredients until homogeneous.
8. Weighing 150 mg homogeneous suspension, and filling into the same hard gelatin capsule.
9. Allowing the formulation to cool down to the room temperatures.
   Analytical Testing-Dissolution Profile of Formulation 3

The drug release profile of Formulation 3 of zaleplon was evaluated by US Pharmacopoeia XXIII, Method I, in a basket apparatus at 50 rpm in 900 mL of 0.1N HCl solution maintaining at 37° C. for 2 hours. After 2 hours, the acidic medium is decanted. 900 mL of simulated intestinal fluid pre-heated at 37° C. is added into each vessel to continue the dissolution study for another 4 hours. At predetermined time intervals, samples are collected. Zaleplon concentrations in the collected samples are assayed using a HPLC method equipped with an UV detector.

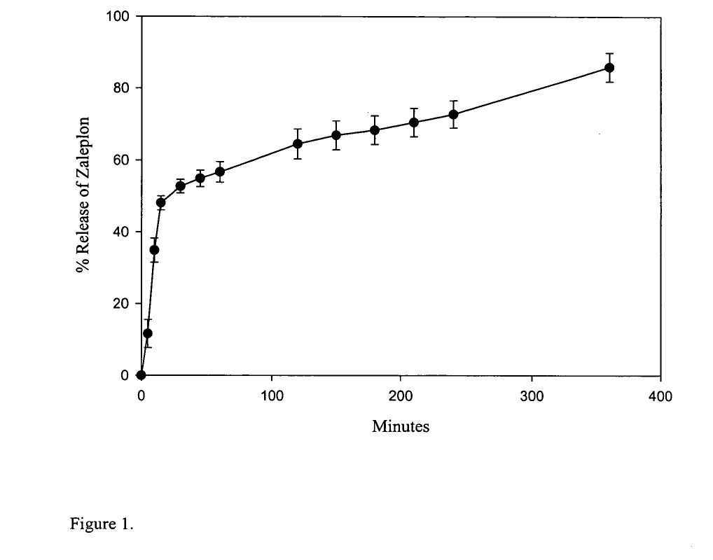

As shown in FIG. 1, a fast release of zaleplon was observed initially for Formulation 3. A delayed and prolonged release profile was then observed after the dissolution medium is switched to the modified simulated intestinal fluids.

### Example 4

Pilot Pharmacokinetic Study in Human

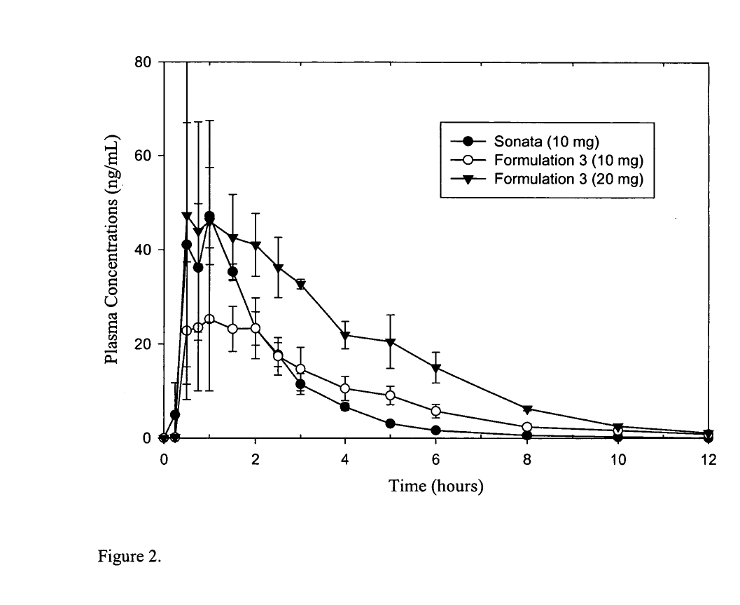

A pilot pharmacokinetic study was conducted in human volunteers. It was a cross-over study comparing the formulations of Formulation 3 and Sonata under fasted condition on 3 healthy volunteers. Blood samples were withdrawn at a predetermined time schedule. Zaleplon concentrations in blood were assayed using a LC/MS/MS system. Plasma concentrations versus time profiles of zaleplon are shown in FIG. 2.

Oral bioavailability is assessed by measuring AUC or Cₘₐₓ, both parameters are well known in the art. AUC is a determination of the area under the curve plotting the serum or plasma concentration of drug along the Y-axis against time along the X-axis. Cₘₐₓ is an abbreviation for the maximum drug concentration achieved in the serum or plasma of the test subject. The pharmacokinetic parameters of the human study are summarized in Table 1.

TABLE 1
Pharmacokinetic Parameters of Zaleplon from a Human Study

| Pharmacokinetic | Sonata | Formulation 3 | Formulation 3 |
|---|---|---|---|
| Parameters | (10 mg) | (10 mg) | (20 mg) |
| Tₘₐₓ (hr) | 0.81 ± 0.29 | 1.17 ± 0.76 | 1.17 ± 0.76 |
| t_{1/2} (hr) | 1.49 ± 0.15 | 2.61 ± 0.42 | 1.61 ± 0.18 |
| Cₘₐₓ(ng/mL) | 56.3 ± 14.6 | 33.3 ± 6.8 | 98.8 ± 0.3 |
| AUC (ng · hr/mL) | 98.7 ± 8.1 | 101.1 ± 20.4 | 208.1 ± 22.3 |

What is claimed is:

1. A pharmaceutical composition comprising a hypnotic agent in a controlled release dosage form that contains at least one immediate release element and at least one delayed release element.

2. The composition as in claim 1, wherein the hypnotic agent is selected from the group consisting of zaleplon, zopiclon and zolpidem or a salt, solvate or hydrate thereof.

3. The composition as in claim 1, wherein the controlled release dosage form is a capsule.

4. The composition as in claim 1, wherein the immediate release element comprises a first hot-melt excipient that is dissolved in a media of pH<5 and in liquid form at temperatures above 35° C.

5. The composition as in claim 4, wherein the first hot-melt excipient is selected from Vitamin E TPGS, Gelucires, and Polyethylene glycols (PEGs), PEG-1500, Weobee and its derivatives, Witepsol and its derivatives, and Tweens.

6. The composition as in claim 1, wherein the delayed release element comprises a second hot-melt excipient that is dissolved in a media of pH≧5 and in liquid form at temperatures above 35° C.

7. The composition as in claim 6, wherein the second hot-melt excipient is selected from sodium alginate, Eudragit, and Polyethylene glycols (PEGs), PEG-1500, and Gelucire, et al.

8. A method of treating sleeping disorders in a subject, the method comprising administering an effective amount of the pharmaceutical composition according to claim 1.

9. A method for the preparation of a controlled release capsule formulation of hypnotic agent, the method comprising filling into the capsule at least one immediate release formulation element and at least one delayed release formulation element.
